(19) United States
(12) Patent Application Publication
(10) Pub. No.: US 2014/0188103 A1
(43) Pub. Date: Jul. 3, 2014

(54) Methods and Apparatus for Neuromodulation Utilizing Optical-Acoustic Sensors

(71) Applicant: Volcano Corporation, San Diego, CA (US)

(72) Inventor: Bret C. Millett, Folsom, CA (US)

(73) Assignee: Volcano Corporation, San Diego, CA (US)

(21) Appl. No.: 14/139,523

(22) Filed: Dec. 23, 2013

Related U.S. Application Data
(60) Provisional application No. 61/747,939, filed on Dec. 31, 2012.

(51) Int. Cl. A61B 18/14; A61F 7/12

(52) U.S. Cl. CPC A61B 18/1492; A61F 7/12
USPC 606/34; 607/113

(57) ABSTRACT

A thermal neuromodulation apparatus, system, and methods for the ablative and non-ablative application of thermal energy to the nerves of a patient are disclosed. The thermal neuromodulation apparatus includes an elongated, hollow body configured to traverse the tortuous intravascular pathways of the renal vasculature and includes an expandable structure bearing electrodes and configured to selectively apply thermal energy via electric fields to the renal nerves through a vessel wall. The thermal neuromodulation apparatus may also include optical-acoustic sensors and an imaging apparatus to obtain data from the treatment area before, during, and after neuromodulation to monitor and/or control the neuromodulation process.

### CROSS-REFERENCE TO RELATED APPLICATIONS

The present application claims priority to and the benefit of U.S. Provisional Patent Application No. 61/747,939, filed Dec. 31, 2012, which is hereby incorporated by reference in its entirety.

### TECHNICAL FIELD

Embodiments of the present disclosure relate generally to the field of medical devices and, more particularly, to an apparatus, systems, and methods for achieving intravascular neuromodulation.

### BACKGROUND

Hypertension and its associated conditions, chronic heart failure (CHF) and chronic renal failure (CRF), constitute a significant and growing global health concern. Current therapies for these conditions span the gamut covering non-pharmacological, pharmacological, surgical, and implanted device-based approaches. Despite the vast array of therapeutic options, the control of blood pressure and the efforts to prevent the progression of heart failure and chronic kidney disease remain unsatisfactory.

Blood pressure is controlled by a complex interaction of electrical, mechanical, and hormonal forces in the body. The main electrical component of blood pressure control is the sympathetic nervous system (SNS), a part of the body's autonomic nervous system, which operates without conscious control. The sympathetic nervous system connects the brain, the heart, the kidneys, and the peripheral blood vessels, each of which plays an important role in the regulation of the body's blood pressure. The brain plays primarily an electrical role, processing inputs and sending signals to the rest of the SNS. The heart plays a largely mechanical role, raising blood pressure by beating faster and harder, and lowering blood pressure by beating slower and less forcefully. The blood vessels also play a mechanical role, influencing blood pressure by either dilating (to lower blood pressure) or constricting (to raise blood pressure).

The kidneys play a central electrical, mechanical and hormonal role in the control of blood pressure. The kidneys affect blood pressure by signaling the need for increased or lowered pressure through the SNS (electrical), by filtering blood and controlling the amount of fluid in the body (mechanical), and by releasing key hormones that influence the activities of the heart and blood vessels to maintain cardiovascular homeostasis (hormonal). The kidneys send and receive electrical signals from the SNS and thereby affect the other organs related to blood pressure control. They receive SNS signals primarily from the brain, which partially control the mechanical and hormonal functions of the kidneys. At the same time, the kidneys also send signals to the rest of the SNS, which can boost the level of sympathetic activation of all the other organs in the system, effectively amplifying electrical signals in the system and the corresponding blood pressure effects. From the mechanical perspective, the kidneys are responsible for controlling the amount of water and sodium in the blood, directly affecting the amount of fluid within the circulatory system. If the kidneys allow the body to retain too much fluid, the added fluid volume raises blood pressure. Lastly, the kidneys produce blood pressure regulating hormones including renin, a hormone that activates a cascade of events through the renin-angiotensin-aldosterone system (RAAS). This cascade, which includes vasoconstriction, elevated heart rate, and fluid retention, can be triggered by sympathetic stimulation. The RAAS operates normally in non-hypertensive patients but can become overactive among hypertensive patients. The kidney also produces cytokines and other neurohormones in response to elevated sympathetic activation that can be toxic to other tissues, particularly the blood vessels, heart, and kidney. As such, overactive sympathetic stimulation of the kidneys may be responsible for much of the organ damage caused by chronic high blood pressure.

Thus, overactive sympathetic stimulation of the kidneys plays a significant role in the progression of hypertension, CHF, CRF, and other cardio-renal diseases. Heart failure and hypertensive conditions often result in abnormally high sympathetic activation of the kidneys, creating a vicious cycle of cardiovascular injury. An increase in renal sympathetic nerve activity leads to the decreased removal of water and sodium from the body, as well as increased secretion of renin, which leads to vasoconstriction of blood vessels supplying the kidneys. Vasoconstriction of the renal vasculature causes decreased renal blood flow, which causes the kidneys to send afferent SNS signals to the brain, triggering peripheral vasoconstriction and increasing a patient's hypertension. Reduction of sympathetic renal nerve activity, e.g., via renal neuromodulation or denervation of the renal nerve plexus, may reverse these processes.

Efforts to control the consequences of renal sympathetic activity have included the administration of medications such as centrally acting sympatholytic drugs, angiotensin converting enzyme inhibitors and receptor blockers (intended to block the RAAS), diuretics (intended to counter the renal sympathetic mediated retention of sodium and water), and beta-blockers (intended to reduce renin release). The current pharmacological strategies have significant limitations, including limited efficacy, compliance issues, and side effects.

While the existing treatments have been generally adequate for their intended purposes, they have not been entirely satisfactory in all respects. The catheters, systems, and associated methods of the present disclosure overcome one or more of the shortcomings of the prior art.

### SUMMARY

In one exemplary embodiment, the present disclosure describes an apparatus for intravascular thermal neuromodulation, comprising an elongate, hollow body, and expandable structure, at least one electrode and at least one imaging component. The elongate, hollow body includes a proximal portion and a distal portion including a distal tip. The body is configured to have an unexpanded condition wherein the distal portion and the distal tip are in contact with each other and an expanded condition wherein the distal portion and the distal tip are spaced apart from each other. The expandable structure is configured to have an expanded condition and an unexpanded condition, and the expandable structure is disposed in an unexpanded condition within the distal portion and proximal to the distal tip. The expandable structure includes at least one support arm. The at least one electrode and the at least one imaging component are positioned on the at least one support arm of the expandable structure. In a further aspect, the imaging component is an optical-acoustic sensor and the arm includes at least one optical fiber.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a block diagram illustrating the pathophysiologic connection between the sympathetic nervous system, the brain, the peripheral vasculature, and the kidneys.

FIG. 2 is a schematic diagram illustrating the thermal basket catheter in an expanded condition according to one embodiment of the present disclosure positioned within the renal anatomy.

FIG. 3 is a schematic diagram illustrating a cross-sectional view of a segment of a renal artery.

FIG. 4a is a schematic diagram illustrating a perspective view of a portion of the renal nerve plexus overlying a segment of a renal artery.

FIG. 4b is a schematic diagram illustrating a perspective view of a portion of the renal nerve plexus overlying a segment of an atherosclerotic renal artery.

FIG. 4c is a schematic diagram illustrating a perspective view of a portion of the renal nerve plexus overlying a segment of a renal artery.

FIG. 5 is a schematic illustration of a thermal neuromodulation system including a thermal basket catheter according to one embodiment of the present disclosure.

FIGS. 6 and 7 are illustrations of a side view of a portion of an optical-acoustic sensor in a first mode and a second mode.

FIG. 8 is an illustration of a single optical fiber having multiple optical-acoustic sensing regions.

FIGS. 9a and 9b are illustrations of a partial cross-sectional side view of the expandable structure in a non-deployed and unexpanded condition and a deployed, expanded condition according to one embodiment of the present disclosure.

FIG. 10a is an illustration of a perspective side view of a thermal basket according to one aspect, along with FIG. 10b showing a cross-section of one of the arms of the basket.

FIG. 11 is an illustration of a partially cross-sectional perspective view of a portion of the thermal basket catheter pictured in FIG. 18a in an expanded condition positioned within a vessel according to one embodiment of the present disclosure.

FIG. 12 is an illustration of a partially cross-sectional perspective view of a portion of a thermal basket catheter in an expanded condition positioned within a vessel according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. For the sake of brevity, however, the numerous iterations of these combinations will not be described separately. For simplicity, in some instances the same reference numbers are used throughout the drawings to refer to the same or like parts.

The present disclosure relates generally to an apparatus, systems, and methods of using thermal energy neuromodulation for the treatment of various cardiovascular diseases, including, by way of non-limiting example, hypertension, chronic heart failure, and/or chronic renal failure. In some instances, embodiments of the present disclosure are configured to deliver thermal energy to the renal nerve plexus to decrease renal sympathetic activity. Renal sympathetic activity may worsen symptoms of hypertension, heart failure, and/or chronic renal failure. In particular, hypertension has been linked to increased sympathetic nervous system activity stimulated through any of four mechanisms, namely (1) increased vascular resistance, (2) increased cardiac rate, stroke volume and output, (3) vascular muscle defects, and/or (4) sodium retention and renin release by the kidney. As to this fourth mechanism in particular, stimulation of the renal sympathetic nervous system can affect renal function and maintenance of homeostasis. For example, an increase in efferent renal sympathetic nerve activity may cause increased renal vascular resistance, renin release, and sodium retention, all of which exacerbate hypertension.

Thermal neuromodulation by either intravascular heating or cooling may decrease renal sympathetic activity by disabling the efferent and/or afferent sympathetic nerve fibers that surround the renal arteries and innervate the kidneys through renal denervation, which involves selectively disabling renal nerves within the sympathetic nervous system (SNS) to create at least a partial conduction block within the SNS. Thermal neuromodulation is due at least in part to the thermally-induced alterations of the neural structures themselves. Additionally or alternatively, the thermal neuromodulation may be due at least in part to the thermally-induced alteration of vascular structures, e.g. arteries, arterioles, capillaries, and/or veins, which perfuse the neural fibers surrounding the target area. Additionally or alternatively, the thermal neuromodulation may be due at least in part to the electroporation of the target neural fibers.

Although the following description is provided in relation to neuromodulation of the renal nerves, it is contemplated that the disclosed devices and methods have application in many different systems of the body. As an additional example, the disclosed systems can be utilized in carotid body baroreceptor ablation or aortic baroreceptor ablation to achieve neuromodulation. Still further, sensor data for the following described system can be utilized to provide tissue characterization information to the user. Further details of using a sensing systems in this manner is disclosed in co-pending application entitled “Device, System and Method for Imaging and Tissue Characterization of Ablated Tissue,” Ser. No. 61/745,476 filed Dec. 12, 2012, as well as co-pending application entitled “Methods and Apparatus for Renal Neuromodulation,” Ser. No. 13/458,856 filed Apr. 27, 2012, each of which is incorporated by reference in their entirety herein.

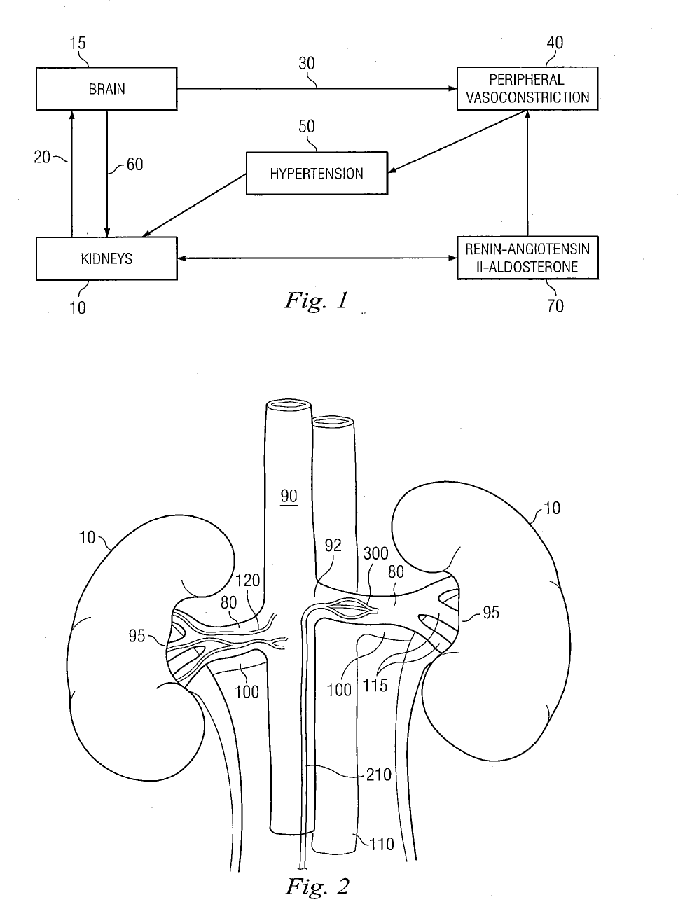

FIG. 1 illustrates the role of the kidneys 10 and renal nerve activity in the progression of hypertension. Several forms of renal injury or stress may induce activation of the renal afferent (from the kidney 10 to the brain 15 or the other kidney) signals 20. For example, renal ischemia, a reduction in stroke volume or renal blood flow, or an abundance of adenosine enzyme may trigger activation of renal afferent nerve activity 20. Increased renal afferent nerve activity 20 results in increased systemic sympathetic activation 30 and peripheral vasoconstriction (narrowing) 40 of blood vessels. Increased vasoconstriction results in increased resistance of blood vessels, which results in hypertension 50. Increased renal efferent (from the brain 15 to the kidney 10) nerve activity 60 results in further increased afferent renal nerve activity 20 and activation of the RAAS cascade 70, inducing increased secretion of renin, sodium retention, fluid retention, and reduced renal blood flow through vasoconstriction. The RAAS cascade 70 also contributes to systemic vasoconstriction of blood vessels 40, thereby exacerbating hypertension 50. In addition, hypertension 50 often leads to vasoconstriction and atherosclerotic narrowing of blood vessels supplying the kidneys 10, which causes renal hypoperfusion and triggers increased renal afferent nerve activity 20. In combination this cycle of factors results in fluid retention and increased workload on the heart, thus contributing to the further cardiovascular and cardio-renal deterioration of the patient. Therefore, FIG. 1 suggests how modulation of afferent and efferent sympathetic renal nerve activity may benefit patients with cardiovascular and cardio-renal diseases, including hypertension.

Renal denervation, which affects both the electrical signals going into the kidneys (efferent sympathetic activity 60) and the electrical signals emanating from them (afferent sympathetic activity 20), has the potential to impact the mechanical and hormonal activities of the kidneys 10 themselves, as well as the electrical activation of the rest of the SNS. Blocking efferent sympathetic activity 60 to the kidney may alleviate hypertension 50 and related cardiovascular diseases by reversing fluid and salt retention (augmenting natriuresis and diuresis), thereby lowering the fluid volume and mechanical load on the heart, and reducing inappropriate renin release, thereby halting the deleterious hormonal RAAS cascade 70 before it starts.

By blocking afferent sympathetic activity 20 from the kidney 10 to the brain 15, renal denervation may lower the level of activation of the whole SNS. Thus, renal denervation may also decrease the electrical stimulation of other members of the sympathetic nervous system, such as the heart and blood vessels, thereby causing additional anti-hypertensive effects. In addition, blocking renal nerves may also have beneficial effects on organs damaged by chronic sympathetic over-activity, because it may lower the level of cytokines and hormones that may be harmful to the blood vessels, kidney, and heart.

Furthermore, because renal denervation reduces overactive SNS activity, it may be valuable in the treatment of several other medical conditions related to hypertension. These conditions, which are characterized by increased SNS activity, include left ventricular hypertrophy, chronic renal disease, chronic heart failure, insulin resistance (diabetes and metabolic syndrome), cardio-renal syndrome, osteoporosis, and sudden cardiac death. For example, other benefits of renal denervation may theoretically include: reduction of insulin resistance, reduction of central sleep apnea, improvements in perfusion to exercising muscle in heart failure, reduction of left ventricular hypertrophy, reduction of ventricular rates in patients with atrial fibrillation, abrogation of lethal arrhythmias, and slowing of the deterioration of renal function in chronic kidney disease. Moreover, chronic elevation of renal sympathetic tone in various disease states that exist with or without hypertension may play a role in the development of overt renal failure and end-stage renal disease. Because the reduction of afferent renal sympathetic signals contributes to the reduction of systemic sympathetic stimulation, renal denervation may also benefit other organs innervated by sympathetic nerves. Thus, renal denervation may also alleviate various medical conditions, even those not directly associated with hypertension.

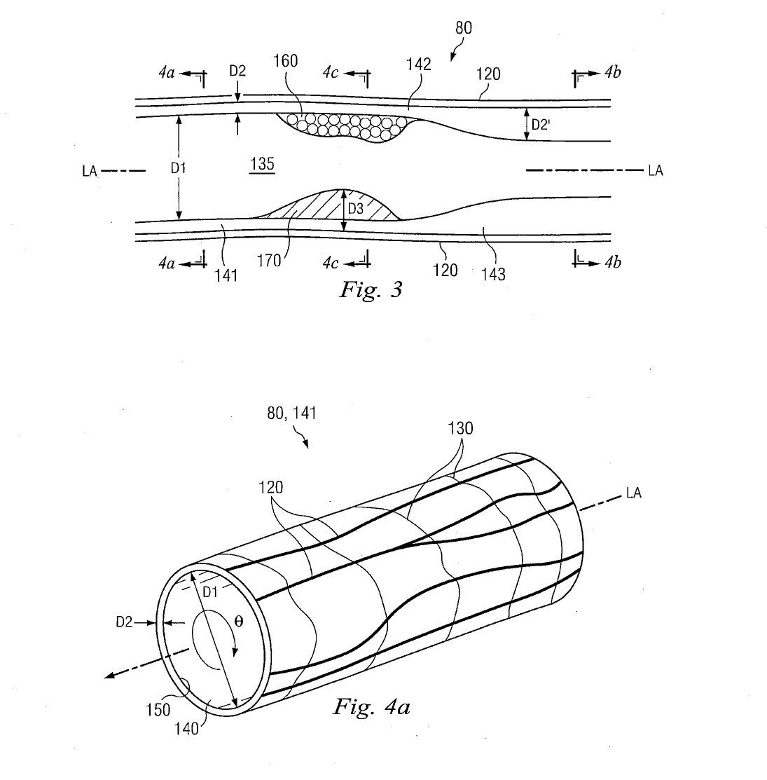

FIG. 2 illustrates a portion of a thermal basket catheter 210 in an expanded condition positioned within the human renal anatomy. The human renal anatomy includes kidneys 10 that are supplied with oxygenated blood by right and left renal arteries 80, which branch off an abdominal aorta 90 at the renal ostia 92 to enter the hilum 95 of the kidney 10. The abdominal aorta 90 connects the renal arteries 80 to the heart (not shown). Deoxygenated blood flows from the kidneys 10 to the heart via renal veins 100 and an inferior vena cava 110. Specifically, the thermal basket catheter 210 is shown extending through the abdominal aorta and into the left renal artery 80. In alternate embodiments, the thermal basket catheter may be sized and configured to travel through the inferior renal vessels 115 as well. The thermal basket catheter 210 will be described in more detail below with respect to FIGS. 9-12.

Left (not shown) and right renal plexi or nerves 120 surround the left and right renal arteries 80, respectively. Anatomically, the renal nerve 120 forms one or more plexi within the adventitial tissue surrounding the renal artery 80. For the purpose of this disclosure, the renal nerve is defined as any individual nerve or plexus of nerves and ganglia that conducts a nerve signal to and/or from the kidney 10 and is anatomically located on the surface of the renal artery 80, parts of the abdominal aorta 90 where the renal artery 80 branches off the aorta 90, and/or on inferior branches of the renal artery 80. Nerve fibers contributing to the plexi 120 arise from the celiac ganglion, the lowest splanchnic nerve, the corticorenal ganglion, and the aortic plexus. The renal nerves 120 extend in intimate association with the respective renal arteries into the substance of the respective kidneys 10. The nerves are distributed with branches of the renal artery to vessels of the kidney 10, the glomeruli, and the tubules. Each renal nerve 120 generally enters each respective kidney 10 in the area of the hilum 95 of the kidney, but may enter in any location where a renal artery 80 or branch of the renal artery enters the kidney.

Proper renal function is essential to maintenance of cardiovascular homeostasis so as to avoid hypertensive conditions. Excretion of sodium is key to maintaining appropriate extracellular fluid volume and blood volume, and ultimately controlling the effects of these volumes on arterial pressure. Under steady-state conditions, arterial pressure rises to that pressure level which results in a balance between urinary output and water and sodium intake. If abnormal kidney function causes excessive renal sodium and water retention, as occurs with sympathetic overstimulation of the kidneys through the renal nerves 120, arterial pressure will increase to a level to maintain sodium output equal to intake. In hypertensive patients, the balance between sodium intake and output is achieved at the expense of an elevated arterial pressure in part as a result of the sympathetic stimulation of the kidneys through the renal nerves 120. Thermal neuromodulation of the renal nerves 120 may help alleviate the symptoms and sequelae of hypertension by blocking or suppressing the efferent and afferent sympathetic activity of the kidneys 10.

FIG. 3 illustrates a segment of the renal artery 80 in greater detail, showing various intraluminal characteristics and intra-to-extraluminal distances that may be present within a single vessel. In particular, the renal artery 80 includes a lumen 135 that extends lengthwise through the renal artery along a longitudinal axis LA. The lumen 135 is a tube-like passage that allows the flow of oxygenated blood from the abdominal aorta to the kidney. The sympathetic renal nerves 120 extend generally within the adventitia (not shown) surrounding the renal artery 80, and include both the efferent (conducting away from the central nervous system) and afferent (conducting toward the central nervous system) renal nerves.

The renal artery 80 includes a first portion 141 having a generally healthy luminal diameter D1 and an intra-to-extraluminal distance D2, a second portion 142 having a narrowed and irregular lumen and an enlarged intra-to-extraluminal distance D3 due to atherosclerotic changes in the form of plaques 160, 170, and a third portion 143 having a narrowed lumen and an enlarged intra-to-extraluminal distance D2′ due to a thickened arterial wall 150. Thus, the intraluminal contour of a vessel, for example, the renal artery 80, may be greatly varied along the length of the vessel. Variable intra-to-extraluminal distances along the length of the vessel may affect the treatment protocols for implementing thermal neuromodulation at different portions of the vessel at least because the amount of thermal energy necessary to travel the intra-to-extraluminal distance to affect neural tissue surrounding the vessel varies with varying intra-to-extraluminal distances. As described further below in relation to FIG. 15, the thermal basket catheters disclosed herein may aid in determining appropriate and effective treatment protocols by pre-treatment, in-treatment, and post-treatment imaging and sensing of various characteristics.

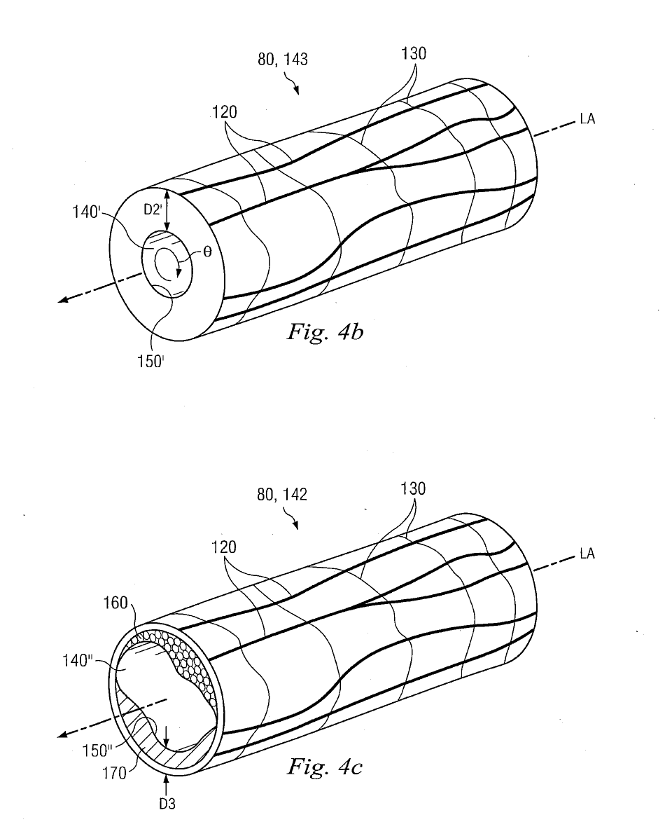

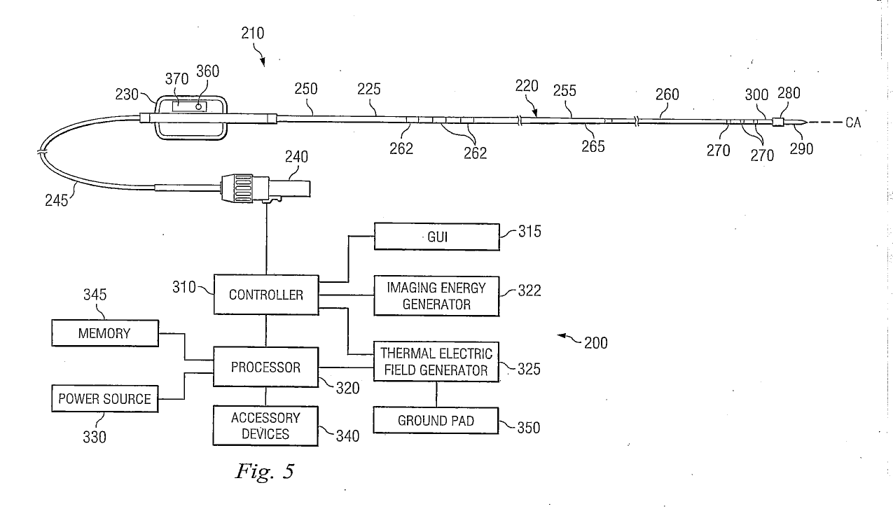

FIGS. 4a, 4b, and 4c illustrate the portions 141, 143, 142, respectively, of the renal artery 80 in perspective view, showing the sympathetic renal nerves 120 that line the renal artery 80. FIG. 4a illustrates the portion 141 of the renal artery 80 including the renal nerves 120, which are shown schematically as a branching network attached to the external surface of the renal artery 80. The renal nerves 120 extend generally lengthwise along the longitudinal axis LA of renal artery 80. In the case of hypertension, the sympathetic nerves that run from the spinal cord to the kidneys 10 signal the body to produce norepinephrine, which leads to a cascade of signals ultimately causing a rise in blood pressure. Neuromodulation of the renal nerves 120 (or renal denervation) removes or diminishes this response and facilitates a return to normal blood pressure.

The renal artery 80 has smooth muscle cells 130 that surround the arterial circumference and spiral around the angular axis θ of the artery. The smooth muscle cells 130 of the renal artery 80 have a longer dimension extending transverse (i.e., non-parallel) to the longitudinal axis LA of the renal artery 80. The misalignment of the lengthwise dimensions of the renal nerves 120 and the smooth muscle cells 130 is defined as “cellular misalignment.” This cellular misalignment of the renal nerves 120 and the smooth muscle cells 130 may be exploited to selectively affect renal nerve cells with a reduced effect on smooth muscle cells.

In FIG. 4a, the first portion 141 of the renal artery 80 includes a lumen 140 that extends lengthwise through the renal artery along the longitudinal axis LA. The lumen 140 is a generally cylindrical passage that allows the flow of oxygenated blood from the abdominal aorta to the kidney. The lumen 140 includes a luminal wall 150 that forms the blood-contacting surface of the renal artery 80. The distance D1 corresponds to the luminal diameter of lumen 140 and defines the diameter or perimeter of the blood flow lumen. A distance D2, corresponding to the wall thickness, exists between the luminal wall 150 and the renal nerves 120. The relatively healthy renal artery 80 may have an almost uniform distance D2 or wall thickness with respect to the lumen 140. The relatively healthy renal artery 80 may decrease substantially regularly in cross-sectional area and volume per unit length, from a proximal portion near the aorta to a distal portion near the kidney.

FIG. 4b illustrates the third portion 143 of the renal artery 80 including a lumen 140′ that extends lengthwise through the renal artery along the longitudinal axis LA. The lumen 140′ includes a luminal wall 150′ which forms the blood-contacting surface of the renal artery 80′. In some patients, the smooth muscle wall of the renal artery is thicker than in other patients, and consequently, as illustrated in FIG. 3b, the lumen of the third portion 143 of the renal artery 80 possesses a smaller diameter relative to the renal arteries of other patients. The lumen 140′, which is smaller in diameter and cross-sectional area than the lumen 140 pictured in FIG. 4a, is a generally cylindrical passage that allows the flow of oxygenated blood from the abdominal aorta to the kidney. A distance D2′ exists between the luminal wall 150′ and the renal nerves 120 that is greater than the distance D2 pictured in FIG. 4a.

FIG. 4c illustrates the diseased second portion 142 of the renal artery 80 including atherosclerotic changes. The second portion 142 includes a lumen 140″ that extends lengthwise through the renal artery along the longitudinal axis LA. Unlike the renal artery of a patient without atherosclerotic changes, as is pictured in FIGS. 4a and 4b, the lumen 140″ is an irregularly-shaped passage that may allow the flow of oxygenated blood from the abdominal aorta to the kidney at a reduced rate because the narrowed lumen creates a reduced cross-sectional area for blood flow. The lumen 140″ includes a luminal wall 150″ which forms the blood-contacting surface of the renal artery 80. The luminal wall 150″ is irregularly shaped by the presence of two atherosclerotic plaques 160, 170. A distance D3 exists between the luminal wall 150″ and the renal nerves 120 that is greater than the distance D2 pictured in FIG. 4a.

Earlier stages of atherosclerotic plaque formation are manifested as “fatty or lipid streaks” on luminal walls. These fatty streaks contain lipid-laden foam cells located in the subendothelial layer of the arterial intima. Additional intracellular and extracellular lipids accumulate at the site of the plaque during later plaque formation stages to cause raised lesions, such as the plaques 160, 170. In addition, smooth muscle and connective tissue cells may migrate into the plaque and proliferate within the plaque. Plaques damage the luminal surface of the artery, thereby weakening the artery and decreasing its elasticity. Luminal damage may also attract additional cells and extracellular materials to accumulate at or near the plaque. Over time, a plaque may calcify. As cells and extracellular materials accumulate, the luminal surface of the artery becomes irregular, as pictured in FIG. 4c, which may lead to the accumulation of blood platelets and thrombus formation. The American Heart Association has recognized several different stages of plaque formation starting from flat lipid streaks, through the visible raised lesions, and ending in a fully occluded artery. As such, atherosclerotic plaque formation is a continuum of events. As the plaques mature, the thickness of the arterial wall, and therefore the distance from the luminal wall to the nerves surrounding the artery, may expand.

In FIG. 4c, the atherosclerotic plaque 160 is a predominantly fatty plaque in the earlier stages of plaque formation. The atherosclerotic plaque 170 is a hardened, calcified plaque in the later stages of plaque formation. The distance D3 extending from the luminal wall 150″ to the renal nerves ranges in thickness along the circumferential and longitudinal span of the plaques 160, 170. Different types of plaques may possess different conductive and impedance properties, thereby affecting the amount, type, and duration of thermal energy that may be required to effectively modulate the nerves overlying the vessels in the region of the plaques.

FIG. 5 illustrates a thermal neuromodulation system 200 that is configured to deliver a thermal electric field to renal nerve fibers in order to achieve renal neuromodulation via heating and/or cooling according to one embodiment of the present disclosure. The system 200 includes a thermal basket catheter 210 comprising an elongate, flexible, tubular body 220 that is configured for intravascular placement and defines an internal lumen 225. The body 220 extends from a handle 230 along a longitudinal axis CA, which is coupled to an interface 240 by an electrical connection 245. The body 220 includes a proximal portion 250, and intermediate portion 255, and a distal portion 260. In FIG. 5, the thermal basket catheter 210 is pictured in an unexpanded condition. The proximal portion 250 may include shaft markers 262 to aid in positioning the catheter in the body of a patient. The intermediate portion 255 may include a guidewire exit port 265 from which a guidewire may emerge. The distal portion 260 may include several radiopaque markers 270, an imaging apparatus 280, and a distal tip 290. In addition, the distal portion 260 comprises an expandable structure 300 (not shown in FIG. 5) in an unexpanded condition within the body 220, located within the distal portion 260 and proximal to the distal tip 290. The imaging apparatus 280 is positioned on a proximal segment of the distal tip 290, which may be axially spaced from the rest of the body 220 along the longitudinal axis CA to reveal the expandable structure 300 in a gradually expanding condition.

The interface 240 is configured to connect the catheter 210 to a patient interface module or controller 310, which may include a guided user interface (GUI) 315. More specifically, in some instances the interface 240 is configured to communicatively connect at least the imaging apparatus 280 and the expandable structure 300 of the catheter 210 to a controller 310 suitable for carrying out intravascular imaging and thermal neuromodulation. The controller 310 is in communication with and performs specific user-directed control functions targeted to a specific device or component of the system 200, such as the thermal basket catheter 210, the imaging apparatus 280, and/or the expandable structure 300.

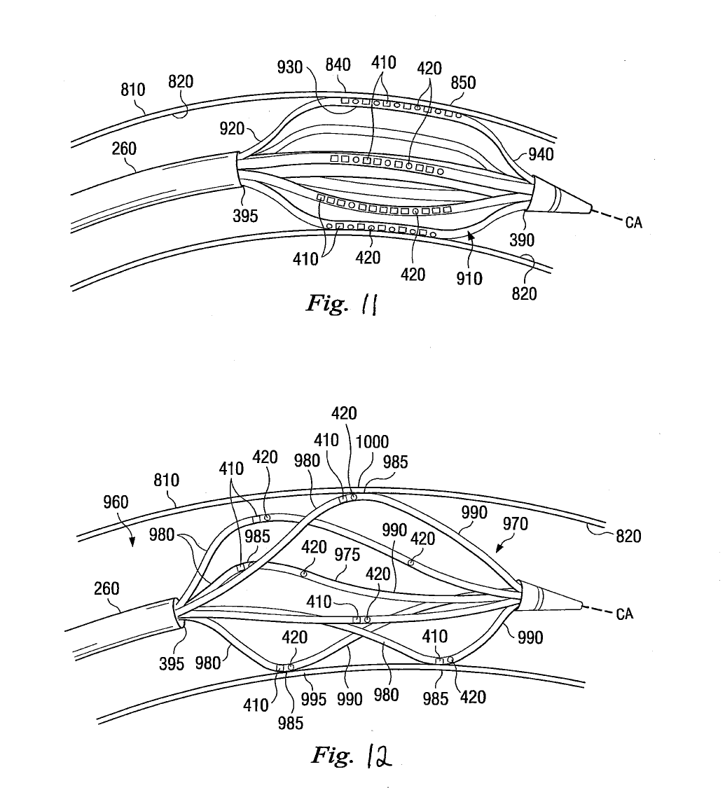

The interface 240 may also be configured to include a plurality of electrical connections and optical connections, each electrically coupled to an electrode on the expandable structure 300 via a dedicated conductor and/or optical fibers extending to optical-acoustic or optical only sensors, respectively, running through the body 220 as described in more detail below with respect to FIG. 11. Such a configuration allows for a specific group or subset of electrodes on the expandable structure 300 to be easily energized with either monopolar or bipolar energy, for example. Similarly, the optical-acoustic sensors positioned on the expandable basket can be energized to interrogate the adjacent tissue structures during the ablation. Such a configuration may also allow the expandable structure 300 to transmit data from any of a variety of sensors via the controller 310 to data display modules such as the GUI 315 and/or the processor 320. The interface 240 may be coupled to the thermal electric field generator 325 via the controller 310, with the controller 310 allowing energy to be selectively directed to the portion of a luminal wall of the renal artery that is engaged by the expandable structure 300 while in an expanded condition.

The controller 310 may be connected to a processor 320, which is typically an integrated circuit with power, input, and output pins capable of performing logic functions, an imaging energy generator 322, and a thermal electric field generator 325. The processor 320 may include any one or more of a microprocessor, a controller, a digital signal processor (DSP), an application specific integrated circuit (ASIC), a field-programmable gate array (FPGA), or equivalent discrete or integrated logic circuitry. In some examples, processor 320 may include multiple components, such as any combination of one or more microprocessors, one or more controllers, one or more DSPs, one or more ASICs, or one or more FPGAs, as well as other discrete or integrated logic circuitry. The functions attributed to processor 320 herein may be embodied as software, firmware, hardware or any combination thereof.

The processor 320 may include one or more programmable processor units running programmable code instructions for implementing the thermal neuromodulation methods described herein, among other functions. The processor 320 may be integrated within a computer and/or other types of processor-based devices suitable for a variety of intravascular applications, including, by way of non-limiting example, thermal neuromodulation and intravascular imaging. The processor 320 can receive input data from the controller 310, from the imaging apparatus 280 and/or the expandable structure 300 directly via wireless mechanisms, or from the accessory devices 340. The processor 320 may use such input data to generate control signals to control or direct the operation of the catheter 210. In some embodiments, the user can program or direct the operation of the catheter 210 and/or the accessory devices 340 from the controller 310 and/or the GUI 315. In some embodiments, the processor 320 is in direct wireless communication with the imaging apparatus 280 and/or the expandable structure 300, and can receive data from and send commands to the imaging apparatus 280 and/or the expandable structure 300.

In various embodiments, processor 320 is a targeted device controller that may be connected to a power source 330, accessory devices 340, a memory 345, and/or the thermal electric field generator 325. In such a case, the processor 320 is in communication with and performs specific control functions targeted to a specific device or component of the system 200, such as the imaging apparatus 280 and/or the expandable structure 300, without utilizing user input from the controller 310. For example, the processor 320 may direct or program the imaging apparatus 280 and/or the expandable structure 300 to function for a period of time without specific user input to the controller 310. In some embodiments, the processor 320 is programmable so that it can function to simultaneously control and communicate with more than one component of the system 200, including accessory devices 330, a power source 340, and/or a thermal electric field generator 325. In other embodiments, the system includes more than one processor and each processor is a special purpose controller configured to control individual components of the system.

The power source 330 may be a rechargeable battery, such as a lithium ion or lithium polymer battery, although other types of batteries may be employed. In other embodiments, any other type of power cell is appropriate for power source 330. The power source 330 provides power to the system 200, and more particularly to the processor 320. The power source 330 may be an external supply of energy received through an electrical outlet. In some examples, sufficient power is provided through on-board batteries and/or wireless powering.

The various peripheral devices 340 may enable or improve input/output functionality of the processor 320. Such peripheral devices 340 include, but are not necessarily limited to, standard input devices (such as a mouse, joystick, keyboard, etc.), standard output devices (such as a printer, speakers, a projector, graphical display screens, etc.), a CD-ROM drive, a flash drive, a network connection, and electrical connections between the processor 320 and other components of the system 200. By way of non-limiting example, a processor may manipulate signals from the imaging apparatus 280 to generate an image on a display device, may coordinate aspiration, irrigation, and/or thermal neuromodulation, and may register the treatment with the image. Such peripheral devices 340 may also be used for downloading software containing processor instructions to enable general operation of the catheter 210, and for downloading software implemented programs to perform operations to control, for example, the operation of any auxiliary devices attached to the catheter 210. In some embodiments, the processor may include a plurality of processing units employed in a wide range of centralized or remotely distributed data processing schemes.

The memory 345 is typically a semiconductor memory such as, for example, read-only memory, a random access memory, a FRAM, or a NAND flash memory. The memory 345 interfaces with processor 320 such that the processor 320 can write to and read from the memory 345. For example, the processor 320 can be configured to read data from the imaging apparatus 280 and write that data to the memory 345. In this manner, a series of data readings can be stored in the memory 345. The processor 320 is also capable of performing other basic memory functions, such as erasing or overwriting the memory 345, detecting when the memory 345 is full, and other common functions associated with managing semiconductor memory.

The controller 310 may be configured to couple the imaging apparatus 280 to an imaging energy generator 322. In embodiments where the imaging apparatus 280 is an IVUS, the imaging energy generator comprises an light generator, such as a controllable laser source. Under the user-directed operation of the controller 310, the imaging energy generator 322 may generate a selected form and magnitude of energy (e.g., a particular energy or light based frequency) best suited to a particular application and best suited to activate a designated optical-acoustic sensor. At least one supply wire (not shown) passing through the body 220 and the interface 240 connects the imaging apparatus 280 to the imaging energy generator 322. The user may use the controller 130 to initiate, terminate, and adjust various operational characteristics of the imaging energy generator 318.

The thermal electric field generator 325 may be configured to produce thermal energy, e.g. RF energy, that may be directed to the expandable structure 300 when it assumes an expanded condition. Under the control of the user or an automated control algorithm in the processor 320, the generator 325 generates a selected form and magnitude of thermal energy. The generator 325 may be utilized with any of the thermal basket catheters described herein for delivery of a thermal electric field with the desired field parameters, i.e., parameters sufficient to thermally induce renal neuromodulation via heating, cooling, and/or other mechanisms such as electroporation. It should be understood that the thermal basket catheters described herein may be electrically connected to the generator 325 even through the generator 325 is not explicitly shown or described with respect to each embodiment. The user may direct whether the expandable structure 300 is energized with monopolar or bipolar RF energy by using the controller 310 or programming the processor 320.

In the pictured embodiment, the generator 325 is located external to the patient. In other embodiments, the generator 325 may be positioned internal to the patient. In alternative embodiments, the generator may additionally comprise or may be substituted with an alternative thermal energy generator, such as, by way of non-limiting example, a thermoelectric generator for heating and/or cooling (e.g., a Peltier device) or a thermal fluid injection system for heating and/or cooling. For embodiments that provide for the delivery of a monopolar electric field via an electrode on the expandable structure 300, a neutral or dispersive ground pad or electrode 350 can be electrically connected to the generator 325. The control and direction of the energy supplied by the generator 325 will be described in further detail with respect to FIGS. 13 and 15.

FIG. 5 illustrates the thermal basket catheter 210 in an unexpanded condition according to one embodiment of the present disclosure. The thermal basket catheter includes the expandable structure 300 in an unexpanded condition positioned within the distal portion 260. As described above, the body 220 is an elongate flexible tube that defines the lumen 225 and the longitudinal axis of the catheter CA. The body 220 is configured to flex in a substantial fashion to traverse tortuous intravascular pathways and gain entrance to the renal arteries. The lumen 225 may be used for the delivery of thermal energy, for sensing various characteristics, and for imaging the vascular and neural anatomy. The lumen 225 may also be used as an access lumen for a guidewire. In some embodiments, the lumen 225 may be used for irrigation of a vessel lumen and aspiration of cellular debris, such as plaque material. In some embodiments, the body 220 includes more than one lumen. The lumen 225 will be described in further detail below with respect to FIGS. 8-10.

As described above, the proximal portion 250 may include shaft markers 262 disposed along the body of the catheter 210 that aid in positioning the catheter in the body of a patient. The shaft markers 262 may be positioned a specific distance from each other and comprise a measurement scale reflecting the distance of the marker 262 from the expandable structure 300. The proximal portion 250 may include any number of shaft markers 262 positioned a fixed distance away from the expandable structure 300 associated with a range of expected distances from the patient's skin surface at the point of catheter entry to the desired zone of thermal neuromodulation. For example, the shaft markers may be positioned, by way of non-limiting example, 1 millimeter from each other, 1 centimeter from each other, and/or 1 inch from each other. After initially positioned the expandable structure within the target vessel for neuromodulation, the user may utilize the shaft markers 262 to knowledgeably shift or reposition the catheter 210 along the intravascular target vessel to apply thermal energy at desired intervals along the target vessel before, after, or without employing imaging guidance. By noting the measurement and/or change in measured distance indicated by the shaft markers located immediately external to the patient's body as the catheter 210 is shifted, the user may determine the approximate distance and axial direction the expandable structure 300 has shifted within the patient's vasculature. In addition, the user may use the measurement and/or change in measured distance indicated by the shaft markers located immediately external to the patient's body to cross reference the intravascular position of the expandable structure 300 indicated by intravascular imaging. In some embodiments, the shaft markers 262 may be radiopaque or otherwise visible to imaging guidance. Other embodiments may lack shaft markers.

The radiopaque markers 270 are spaced along the distal portion 260 at specific intervals from each other and at a specific distance from the distal tip 290. The radiopaque markers 270 may aid the user in visualizing the path and ultimate positioning of the catheter 210 within the vasculature of the patient. In addition, the radiopaque markers 270 may provide a fixed reference point for co-registration of various imaging modalities and treatments, including by way of non-limiting example, external imaging including angiography and fluoroscopy, imaging by the imaging apparatus 280, and thermal neuromodulation by the expandable structure 300. Other embodiments may lack radiopaque markers.

In the pictured embodiment, the imaging apparatus 280 is an intravascular ultrasound (IVUS) apparatus. More specifically, the imaging apparatus 280 pictured in FIG. 5 represents an ultrasound transducer array formed from a plurality of optical-acoustic sensing elements. In one embodiment, the transducer array includes 32 elements, while in others it can include 64, 96 or 128 sensing elements. A bundle of optical fibers interconnects the transducer array with the optical source positioned outside of the body. The entire IVUS apparatus may extend through the body 220 and include all the components associated with an IVUS module. The imaging apparatus 280 of the pictured embodiment may utilize any IVUS configuration that allows at least a portion of the body 220 to be introduced over a guidewire. For example, in some instances, the imaging apparatus 280 utilizes an array of transducers (e.g., 32, 64, 128, or other number transducers) disposed circumferentially about the central lumen 225 of the body 220 in a fixed orientation. In other embodiments, the IVUS portion 280 is a rotational IVUS system having only a single optical-acoustic ultrasonic transducer assembly. In some instances, the imaging apparatus 280 includes components such as transmitters and receivers similar or identical to those found in U.S. Pat. Nos. 7,245,789; 6,659,957 and U.S. application Ser. No. 12/571,724, each of which is hereby incorporated by reference in its entirety. Still further, in some embodiments, the sensors include optical pressure sensors. U.S. Pat. Nos. 7,689,071; 8,151,648 and U.S. application Ser. No. 13/415,514, disclose optical pressure sensors in detail and are herein incorporated by reference in their entirety.

In alternate embodiments, the imaging apparatus 280 may be or include, by way of non-limiting example, any of grey-scale IVUS, forward-looking IVUS, rotational IVUS, phased array IVUS, solid state IVUS, optical-acoustic IVUS, optical coherence tomography, or virtual histology. It is understood that, in some instances, wires and optical fibers associated with the imaging apparatus 280 extend along the length of the elongated tubular body 220 through the handle 230 and along electrical connection 245 to the interface 240 such that signals from the imaging apparatus 280 can be communicated to the controller 310. In some instances, the imaging apparatus 280 communicates wirelessly with the controller 310 and/or the processor 320.

In alternate embodiments, the imaging apparatus 280 may work in cooperation with or be substituted by an independent imaging catheter that is threaded through the lumen 225 of the catheter 210. In such embodiments, the independent imaging catheter may be axially moveable and rotational within the body 220 such that the imaging components of the imaging catheter may be positioned in a multitude of places along the longitudinal axis CA relative to the expandable structure 300. For example, a distal tip of the imaging catheter may be positioned proximal, within, or distal to the expandable structure 300 to gather image data about the surrounding tissue. In an embodiment where the imaging catheter is positioned within the expandable structure, the expandable structure may be constructed of translucent material or material that does not interfere with the data collection of the imaging catheter.

With reference to FIG. 5, in alternate embodiments, the imaging apparatus 280 may work in cooperation with or be substituted by a central imaging apparatus 355, which may be positioned on an exterior surface of an inner body 490 of the body 220. The central imaging apparatus 355 may be configured to function in substantially the same manner as the imaging apparatus 280.

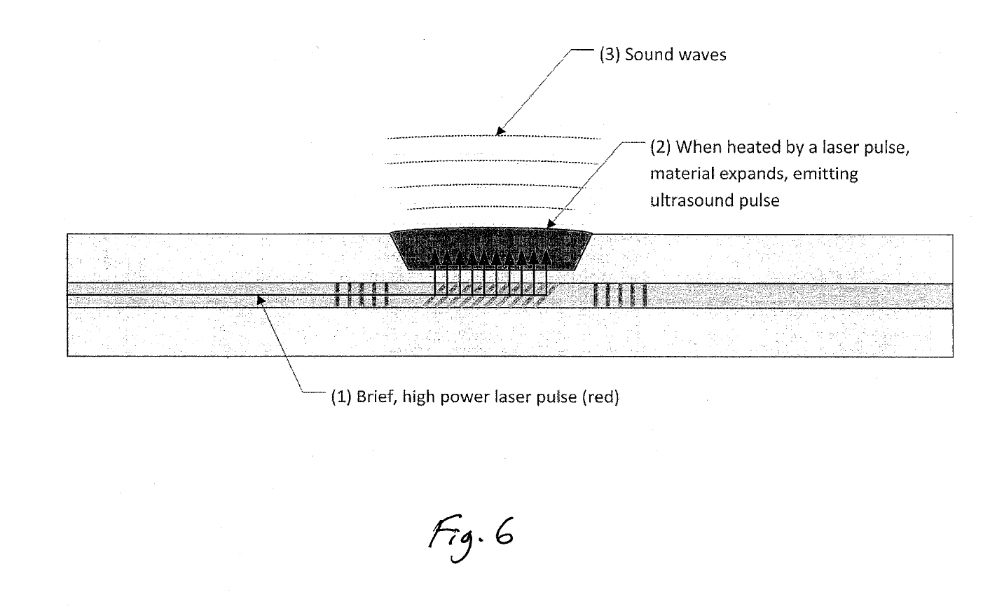

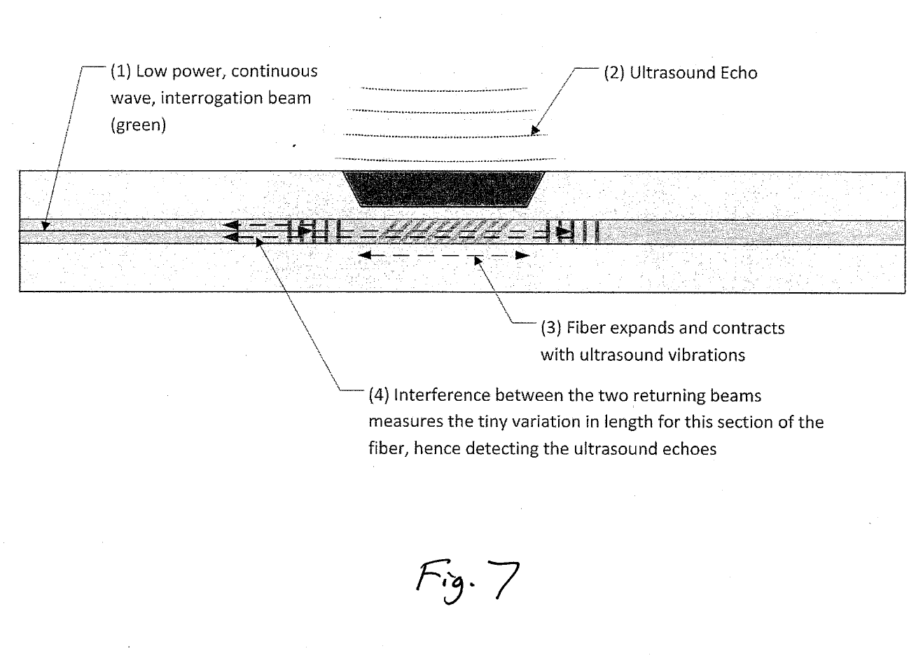

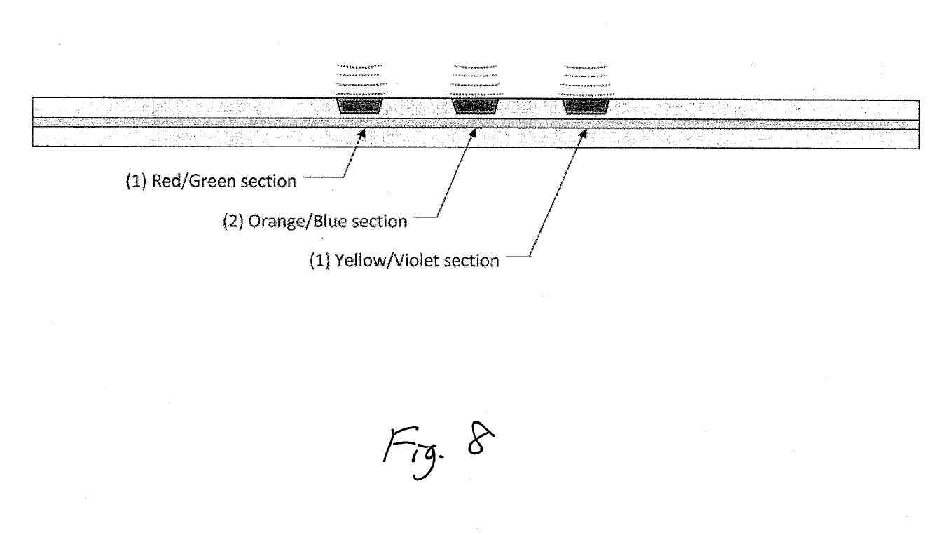

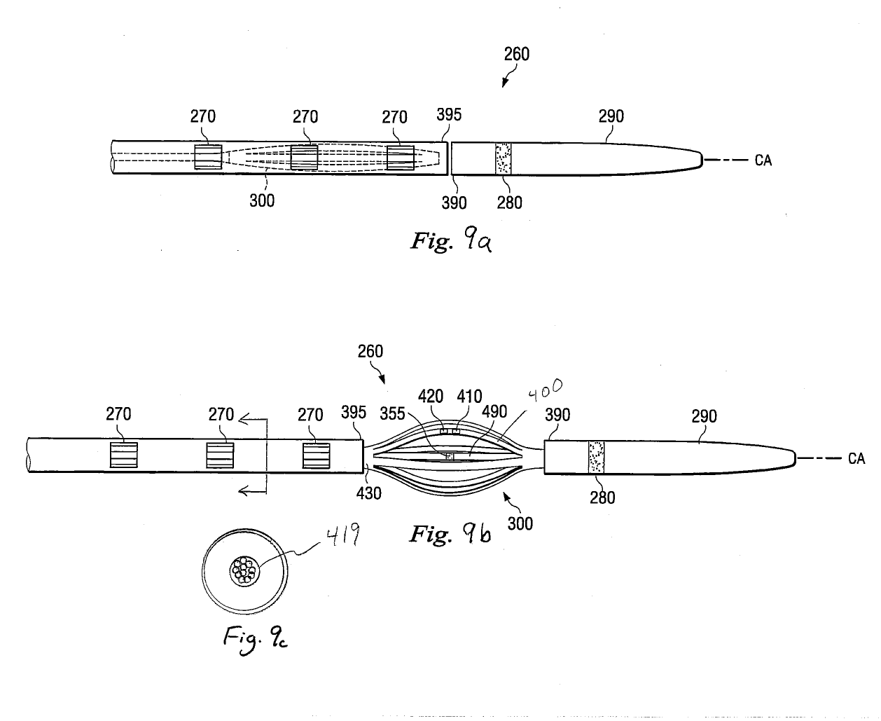

The proximal portion 250 of the body 220 connects to the handle 230, which is sized and configured to be securely held and manipulated by a user outside a patient's body. By manipulating the handle 230 outside the patient's body, the user may advance the body 220 of the catheter 210 through an intravascular path (as illustrated, for example, in FIG. 2) and remotely manipulate or actuate the distal portion 260. In the pictured embodiment, the handle 230 includes an elongated, slidable body actuator 360 positioned within an actuator recess 370. The body actuator 360 may be configured as any of a variety of elements, including by way of non-limiting example, a knob, a pin, or a lever, capable of manipulating or actuating the distal portion 260 to reveal the expandable structure 300. The operation of the body actuator 360 will be further described below with respect to FIGS. 6b and 7.

In alternate embodiments, the handle 230 may include a proximal port configured to receive fluid therethrough, thereby permitting the user to irrigate or flush the lumen 225 and/or the expandable structure 300. For example, the proximal port may include a Luer-type connector capable of sealably engaging an irrigation device such as a syringe. Image guidance using the imaging apparatus 280 or external imaging, e.g., radiographic, CT, or another suitable guidance modality, or combinations thereof, can be used to aid the user's manipulation of the catheter 210. In the pictured embodiment, the body 220 is integrally coupled to the handle 230. In other embodiments, the body 220 may be detachably coupled to the handle 230, thereby permitting the body 220 to be replaceable.

The catheter 210, or the various components thereof, may be manufactured from a variety of materials, including, by way of non-limiting example, plastics, polytetrafluoroethylene (PTFE), polyether block amide (PEBAX), thermoplastic, polyimide, silicone, elastomer, metals, such as stainless steel, titanium, shape-memory alloys such as Nitinol, and/or other biologically compatible materials. In addition, the catheter 210 may be manufactured in a variety of lengths, diameters, dimensions, and shapes. For example, in some embodiments the elongated body 220 may be manufactured to have length ranging from approximately 115 cm-155 cm. In one particular embodiment, the elongated body 220 may be manufactured to have length of approximately 135 cm. In some embodiments, the elongated body 220 may be manufactured to have a transverse dimension ranging from about 1 mm-2.67 mm (3 Fr-8 Fr). In one embodiment, the elongated body 200 may be manufactured to have a transverse dimension of 2 mm (6 Fr), thereby permitting the catheter 210 to be configured for insertion into the renal vasculature of a patient. These examples are provided for illustrative purposes only, and are not intended to be limiting.

FIGS. 6 and 7 illustrate an optical-acoustic sensor formed on an optical fiber. As indicated in FIG. 6, a high energy pulsed laser is transmitted down the fiber and reflected outward by the 45 degree Bragg Grating. The reflected light heats the overlying material to cause an ultrasonic pulse to be generated. In FIG. 7, interference from reflected ultrasonic pulses causes interference in a continuous interrogation beam of a different frequency. Based on the interference, the ultrasonic echo can be detected. By using different frequencies for the high energy pulse and selective Bragg Gratings, a plurality of optical-acoustic sensors can be formed along a single fiber as shown in FIG. 8. As shown in the drawings for illustration purposes, the gratings could be responsive to different wavelengths or colors within the spectrum. While different colors are indicated, it is likely that different frequencies in or near the infrared spectrum would be the likely choice for the high energy pulses. As will be explained more fully below, the multi-sensor fibers can be embedded within moveable components of the system.

FIG. 9a illustrates at least a segment of the distal portion 260 of the thermal basket catheter 210 in an unexpanded condition according to one embodiment of the present disclosure. In some instances, the thermal basket catheter 210 includes components or features similar or identical to those disclosed in U.S. Patent Application Publication No. US2004/0176699, which is hereby incorporated by reference in its entirety. In the pictured embodiment, the distal tip 290 is positioned against the remainder of the body along the longitudinal axis CA, and the expandable structure 300 is compressed within the lumen in an unexpanded condition. The distal portion 260 includes a distal connection part 390, which is the proximal-most part of the distal tip 290, and a proximal connection part 395, which abuts the distal connection part 390 when the catheter 210 is in an unexpanded condition. In the pictured embodiment, the imaging apparatus 280 is positioned distal to the distal connection part 390. As discussed above, in one form, the imaging apparatus 280 comprising an array of optical-acoustic elements. In another form, the imaging apparatus 280 can comprises a single optical-acoustic element that is rotationally moved to generate an image. Additionally or alternatively, the imaging apparatus may be positioned proximal to the proximal connection part 395.

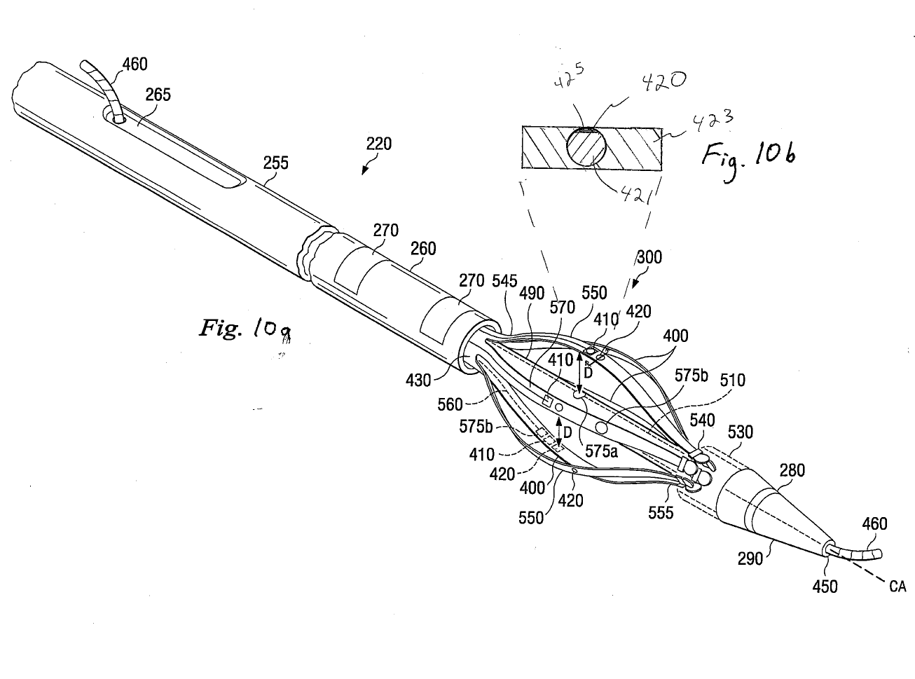

FIG. 9b illustrates at least a segment of the distal portion 260 of the thermal basket catheter 210 in an expanded condition according to one embodiment of the present disclosure. In the pictured embodiment, the distal tip 290 is moved distally away from the remainder of the body along the longitudinal axis CA to allow the expandable structure 300 to emerge from the lumen and assume an expanded condition. Specifically, the distal connection part 390 is separated axially away from the proximal connection part 395 along the axis CA. As further described below, the user may transition the catheter 210 from an unexpanded condition to an expanded condition by manipulating the body actuator 360 within the actuator recess 370 to cause the distal tip 290 to move distally away from the remainder of the body 220. In the pictured embodiment, the expandable structure 300 is shown in a deployed and expanded condition wherein at least one support arm 400 has expanded outwardly. The expandable structure 300 includes six flexible support arms 400. In other embodiments, the expandable structure may include any number of support arms 400. At least one electrode 410 and at least one optical-acoustic sensor 420 may be positioned on at least one of the support arms 400. The at least one electrode 410 and at least one sensor 420 will be described in further detail below with reference to FIGS. 10a and 10b. FIG. 9c shows a cross-section of the shaft illustrating the optical fiber bundle 419 that has fibers extending to the array assembly 280 as well as individual fibers that may extend onto the flexible arms 400 to define optical-acoustic sensors thereon.

The support arms 400 may be manufactured from a variety of biocompatible materials, including, by way of non-limiting example, superelastic or shape memory alloys such as Nitinol, and other metals such as titanium, Elgiloy®, and/or stainless steel. The support arms 400 could also be made of, by way of non-limiting example, polymers or polymer composites that include thermoplastics, resins, carbon fiber, and like materials. In the illustrated embodiment, the support arms 400 are secured to a deployment support member 430, which may be secured to an interior component of the body 220 in a variety of ways, including by way of non-limiting example, adhesively bonded, laser welded, mechanically coupled, or integrally formed. In alternate embodiments, the support arms 400 may be secured to an interior component of the body 220 directly, thereby eliminating the need for a deployment support member 430.

FIG. 10a illustrates the thermal basket catheter 210 in an expanded condition according to one embodiment of the present disclosure wherein the distal tip 290 has been moved axially away from the remainder of the distal portion 260 and at least one of the support arms 400 has expanded outwardly. The support arms 400 may be manufactured in any of a variety of shapes, including by way of non-limiting example, arcuate shapes, bell shapes, smooth shapes, and step-transition shapes. The support arms include a proximal section 545, a medial section 550, and a distal section 555. The proximal section 545 may be capable of coupling the expandable structure 300 to the body 220 or the inner body 490. The medial section 550 is configured to be positioned proximate to or in contact with a vessel luminal wall. The distal section 555 couples each arm 400 to a support arm retainer 540 positioned on an exterior of the inner body 490.

The transverse or cross-sectional profile of the support arms 400 may be manufactured in any of a variety of shapes, including oblong, ovoid, and round. In some embodiments, the cross-sectional profile of the support arm includes rounded or atraumatic edges to minimize damage to an artery or a tubular structure through which the expandable structure 300 may travel.

In one embodiment, the proximal sections 545 of the support arms 400 may be coupled to the deployment support member 430 using an adhesive, such as, by way of non-limiting example, Loctite 3311 adhesive or any other biologically compatible adhesive. In an alternate embodiment, the expandable structure 300 may be manufactured by laser cutting or forming the at least one support arm 50 from a substrate. For example, any number of support arms 400 may be laser cut within a Nitinol tube or cylinder, thereby providing a slotted expandable body. The support arms 400 may be fabricated from a self-expanding material biased such that the medial section 550 expands into contact with the vessel luminal wall upon expanding the catheter 210. In some embodiments, the one or more support arms 400 may be formed in a deployed state as shown in FIG. 10a wherein at least one support arm 400 is flared outwardly from the longitudinal axis CA of the catheter 210.

In the illustrated embodiment, the guidewire lumen 510, capable of receiving the guidewire 460 therein, longitudinally traverses the expandable structure 300. The guidewire lumen 510 is in communication with the guidewire port 450 on the distal portion 260 and guidewire exit slot 265 located on the elongated body 220. In an alternate embodiment, the guidewire lumen 510 may be in communication with the guidewire port 450 on the distal tip 290 and/or a proximal port located on the handle 230 (shown in FIGS. 4 and 5). In the illustrated embodiment, a retainer sleeve 530 is positioned over a distal section of the support arms 400 to provide a transition between the distal tip 290 and the support arms 400. As shown, the retainer sleeve 530 is positioned over the support arm retainers 540, thereby preventing the support arm retainers 540 from contacting the vessel wall 90 and causing trauma to the vessel luminal wall (not shown), damaging the support arm retainers 540, or both. Other embodiments may lack a retainer sleeve.

During manufacture, the at least one support arm 400 is formed to assume a deployed position in a relaxed state as shown in FIG. 12, wherein the medial section 550 of the support arm 400 is flared outwardly a distance D from the longitudinal axis CA of the catheter 210. The application of force to the apex of the medial section 550 of the support arm 400 decreases the curvature of the support arm 400 resulting in a corresponding decrease in the distance D.

The at least one electrode 410 may be positioned on the medial section 550 of at least one of the support arms 400, thereby enabling the electrode 410 and the sensor 420 to contact or approximate the vessel luminal wall. At least one electrode cable 560 connects each electrode 410 to the interface 240 and/or the thermal electric field generator 325. The at least one electrode 410 will be described in further detail below in reference to FIG. 13.

The at least one sensor 420 may be positioned on the medial section 550 of at least one of the support arms 400, thereby enabling the sensor to contact or approximate the vessel luminal wall. In the illustrated embodiment, the sensor is an optical-acoustic sensor as described above. As shown in the FIG. 10b showing a cross-section of arm 400, an optical fiber 421 is embedded within the material 423 forming the arm. An aperture 425 is formed through the material 423 to allow the sensor component to be exposed to the surrounding environment. Although the fiber and sensor are shown embedded within the material, it is contemplated that the fiber and/or sensor may be on the exterior surface of the arm 400 or only partially embedded. In the illustrated embodiment, the fiber 421 can be embedded in a polymer material as the arm 400 is being formed. When the arm is formed of a metal, it may be easier to adhere the optical fiber to the surface of the arm. Still further, while the illustrated sensor is an ultrasound sensor, it is contemplated that other sensors such as optical pressure sensors or light based imaging fibers could be combined with or substituted for the ultrasound sensor.

Referring now to FIGS. 11 and 12, there are shown alternative embodiments of the expandable therapy devices including heating electrodes 410 and sensing devices 420. With respect to FIG. 11, the plurality of sensing locations 420 formed on each arm can be formed by a single fiber having multiple differential frequency Bragg Gratings as discussed above with respect to FIG. 8. In this manner, a single optical fiber can provide a low profile sensing string along the expandable arm 400.

The expandable structure 300 may include at least one ancillary sensor 575 thereon. As shown in FIG. 12, the ancillary sensor 575a may be positioned on an exterior surface of the inner body 490. In the alternative, at least one ancillary sensor 575b may be positioned on at least one support arm 400. Exemplary ancillary sensors 575 include, without limitation, ultrasonic sensors, flow sensors, thermal sensors, blood temperature sensors, electrical contact sensors, conductivity sensors, electromagnetic detectors, pressure sensors, chemical or hormonal sensors, pH sensors, and infrared sensors. For example, in one embodiment the ancillary sensor 575a may comprise a blood sensor positioned on the guidewire lumen 510 in the bloodstream as shown in FIG. 12, thereby permitting the sensors 420 located on the support arms 400 to measure the vessel wall temperature while simultaneously the ancillary sensor 575a measures blood temperature within the vessel. In another embodiment, the ancillary sensor 575b may comprise a pressure sensor positioned on the support arm 400 proximate to the electrode 410 and/or encircling the electrode 410. The ancillary pressure sensor 575b may detect the pressure with which the proximate electrode 410 is contacting the vessel wall, thereby allowing the user to determine whether the electrode 410 is effectively contacting the vessel wall to ensure adequate energy transfer and neuromodulation.

FIG. 11 illustrates the elongated expandable structure 910 in an expanded condition after emerging from the proximal connection part 395 of the distal portion 260. In the pictured embodiment, the intermediate parts 930 of the support arms 400 of the expandable structure 910 have expanded outwardly from the longitudinal axis CA, thereby permitting a majority of the electrodes 410 and the sensors 420 located on the support arms 400 to contact the internal luminal surface 820 of the vessel 810. Using a thermal basket catheter including an elongated expandable structure allows the user to simultaneously apply thermal energy to multiple positions spaced longitudinally along the vessel wall, thereby potentially shortening the duration of the thermal neuromodulation procedure. For example, in the pictured embodiment, the expandable structure 910 may simultaneously apply thermal energy to the vessel wall at a circumferential position 840 and a circumferential position 850, which are spaced longitudinally from each other along the vessel wall of vessel 810. In addition, the spaced optical-acoustic sensors 420 can be utilized to image and characterize adjacent tissue to monitor the ablation process. Thus, each heating electrode can be monitored individually if desired by the user to customize the delivered therapy to correspond to the sensed tissue type, depth or density adjacent the electrode.

FIG. 12 shows a thermal basket catheter 960 including a helical expandable structure 970 positioned within a curved portion 810 of the renal artery 80 (similar to the portion 141 shown in FIG. 2) according to one embodiment of the present disclosure. FIG. 12 illustrates the elongated expandable structure 960 in an expanded condition after emerging from the proximal connection part 395 of the distal portion 260. The thermal basket catheter 970 is substantially identical to the thermal basket catheter 210 except for the differences noted herein. The expandable structure 970 is shaped and configured as an elongated basket comprising support arms 975 that include proximal parts 980, intermediate parts 985, and distal parts 990.

The support arms 975 of the expandable structure 970 include multiple electrodes 410 and sensors 420, at least some of which are positioned along the intermediate parts 985 of the arms 975. In the pictured embodiment, the majority of electrodes 410 and sensors 420 of the expandable structure 960 are clustered on the intermediate parts 985 of the support arms 400. Each arm 975 is shaped and configured to flex at the intermediate part 985, thereby enabling the electrode 420 and/or the sensor 410 to contact an internal luminal surface 820 of the vessel 810. Each proximal part 980 and distal part 990 is shaped and configured to slope from the intermediate part 985 toward the longitudinal axis CA of the catheter 960. The intermediate parts 985, or apex, of each arm 975 in the expanded configuration are staggered longitudinally such that in the expanded condition the intermediate parts align in a generally helical pattern circumferentially extending around the longitudinal axis. In the illustrated embodiments, many arms 975 have a short portion and a long portion that defines the intermediate part 985 therebetween.

In the pictured embodiment, the intermediate parts 985 of the support arms 975 of the helical expandable structure 970 have expanded outwardly from the longitudinal axis CA, thereby permitting a majority of the electrodes 410 and the sensors 420 located on the support arms 400 to contact the internal luminal surface 820 at different linearly-spaced locations along the length of the vessel 810. Such a configuration allows the expandable structure 970 to contact and apply thermal energy to various, linearly-spaced areas along the intraluminal surface, thereby reducing or preventing circumferential thermal injury to a focal, ring-like area of the vessel tissue. In some instances, the expandable structure 970 allows the user and/or processor to apply an energy in a helical or spiral pattern to the intraluminal surface 82-820. Using a thermal basket catheter including a helical expandable structure allows the user to simultaneously apply thermal energy to multiple positions spaced longitudinally along the vessel wall, thereby potentially shortening the duration of the thermal neuromodulation procedure. For example, in the pictured embodiment, the expandable structure 970 may simultaneously apply thermal energy to the vessel wall at a circumferential position 995 and a circumferential position 1000, which are spaced longitudinally from each other along the vessel wall of vessel 810.

It should be appreciated that while several of the exemplary embodiments herein are described in terms of an ultrasonic device, or more particularly the use of IVUS data obtained via optical-acoustic sensors (or a transformation thereof) to render images of a vascular object, the present disclosure is not so limited. Thus, for example, an imaging device using backscattered data (or a transformation thereof) based on ultrasound waves or even electromagnetic radiation (e.g., light waves in non-visible ranges such as Optical Coherence Tomography, X-Ray CT, etc.) to render images of any tissue type or composition (not limited to vasculature, but including other human as well as non-human structures) is within the spirit and scope of the present disclosure.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example, the thermal basket catheter may be utilized anywhere with a patient's vasculature, both arterial and venous, having an indication for thermal neuromodulation. It is understood that such variations may be made to the foregoing without departing from the scope of the present disclosure. Accordingly, it is appropriate that the appended claims be construed broadly and in a manner consistent with the present disclosure.

1. An apparatus for intravascular thermal neuromodulation, comprising:
an elongate, hollow body including a proximal portion and a distal portion, the distal portion including a distal tip, the body configured to have an unexpanded condition wherein the distal portion and the distal tip are in contact with each other, and an expanded condition wherein the distal portion and the distal tip are spaced apart from each other;
an expandable structure configured to have an expanded condition and an unexpanded condition, the expandable structure disposed in an unexpanded condition within the distal portion and proximal to the distal tip, the expandable structure including at least one support arm;
at least one electrode positioned on the at least one support arm of the expandable structure; and
at least one optical-acoustic sensor positioned adjacent the expandable structure.

2. The apparatus of claim 1, wherein the optical-acoustic sensor is positioned on the at least one support arm of the expandable structure.

3. The apparatus of claim 2, wherein further including a plurality of optical-acoustic sensors positioned on the at least one support arm of the expandable structure.

4. The apparatus of claim 1, wherein the optical-acoustic sensor is positioned distally of the expandable structure.

5. The apparatus of claim 1, further comprising an outer sleeve positioned around the elongated, hollow body, the outer sleeve defining a sleeve lumen sized and shaped to receive the expandable structure therein in the unexpanded condition.

6. The apparatus of claim 1, further comprising an imaging apparatus positioned on the body.

7. The apparatus of claim 6, wherein the imaging apparatus is positioned within the expandable structure on the body.

8. The apparatus of claim 1, wherein the expandable structure is configured for placement within a vessel lumen such that the at least one support arm contacts a vessel luminal wall when the expandable structure is in an expanded condition.

9. The apparatus of claim 8, wherein the at least one electrode is positioned on the at least one support arm such that the at least one electrode contacts the vessel luminal wall when the expandable structure is in an expanded condition.

10. The apparatus of claim 9, wherein the at least one electrode is configured to transmit thermal energy through the vessel luminal wall to a renal nerve.

11. A method for thermal modulation of nerves overlying a vessel, comprising:
positioning a thermal neuromodulation apparatus including at least one optical-acoustic sensor positioned adjacent the expandable structure and an expandable structure carrying at least one electrode within a lumen of the vessel;
positioning the thermal neuromodulation apparatus in the vessel;
expanding the expandable structure to enable the at least one electrode to contact a luminal wall proximate the nerves overlying the vessel;
directing thermal energy from the at least one electrode through the luminal wall to the nerves; and
imaging the luminal wall of the vessel and the nerves with the at least one optical-acoustic sensor to obtain image data reflective of the extent of tissue damage.

12. The method of claim 11, further comprising imaging the luminal wall of the vessel with the at least one optical acoustic sensor to obtain image data reflecting structural characteristics and a circumferential wall thickness of the lumen prior to directing the thermal energy to the nerves.

13. The method of claim 12, wherein positioning the thermal neuromodulation apparatus in the vessel includes selecting an optimal intravascular location based on the image data obtained prior to directing the thermal energy to the nerves.

14. The method of claim 11, further comprising modifying the amount and duration of applied thermal energy from the at least one electrode through the luminal wall to the nerves based on the image data reflective of the extent of tissue damage.

15. The method of claim 11, further comprising retracting the expandable structure and withdrawing the thermal neuromodulation apparatus from the vessel.
